(19) United States
(12) Patent Application Publication
(10) Pub. No.: US 2007/0020741 A1
(43) Pub. Date: Jan. 25, 2007

(54) Process for preparing enantiomer-enriched alpha-hydroxycarboxylic acids and amides

(76) Inventors: Karlheinz Drauz, Freigericht (DE); Stefan Buchholz, Hanau (DE); Harald Groger, Hanau (DE)

Correspondence Address: FITCH, EVEN, TABIN & FLANNERY, P. O. BOX 18415, WASHINGTON, DC, 20036, US

(21) Appl. No.: 10/575,386

(22) PCT Filed: Oct. 7, 2004
(86) PCT No.: PCT/EP04/11183

§ 371 Date: Apr. 7, 2006

(30) Foreign Application Priority Data
Oct. 10, 2003 (DE) 103 47 888.4

(51) Int. Cl. C12P 7/40; C12N 9/80

(52) U.S. Cl. 435/136; 435/228

(57) ABSTRACT

The present invention describes an enzymatic process for preparing enantiomer-enriched α-hydroxycarboxylic acids and amides which comprises, in one step, the conversion of a carbonyl compound to the corresponding acid/amides via the intermediate stage of a cyanohydrin. The invention also provides a reaction system operating in such a way and a whole-cell catalyst that is advantageous for use for this reaction.

The present invention relates to a process for preparing enantiomer-enriched α-hydroxycarboxylic acids and amides. In particular, the invention relates to a process wherein, in a first step, a cyanohydrin is generated from cyanide donors, an aldehyde and a ketone in the presence of an oxynitrilase, said cyanohydrin being converted further, in a second step, to the corresponding acid by a nitrilase or nitrile hydratase. The invention further relates to a reaction system operating in such a way, and also to new organisms that are capable of implementing the aforementioned two-stage reaction.

Enantiomer-enriched α-hydroxycarboxylic acids and amides thereof are important synthetic products in the field of organic chemistry. These compounds can be employed successfully as precursor molecules for ligand syntheses, as chiral racemate-resolution agents, or as intermediate products for the preparation of biologically active substances.

The classical synthesis of this type of compounds is generally undertaken by a cyanohydrin reaction with subsequent acid hydrolysis and resolution of racemates via diastereomeric salt formation (Bayer-Walter, Lehrbuch der Organischen Chemie, S. Hirzel Verlag Stuttgart, 22nd edition, p. 555). The hydrolysis may optionally be stopped at the stage of the amides or may be implemented in full as far as the acid.

The preparation of optically active α-hydroxycarboxylic acids has also been obtained hitherto either by the formation of cyanohydrin being carried out in the form of an asymmetric addition of a cyanide donor to an aldehyde in the presence of a chiral catalyst, for example an enzyme such as oxynitrilase, followed by a “classical” hydrolysis, or alternatively by preparation of a racemic cyanohydrin, followed by enantioselective hydrolysis in the presence of a nitrilase. The first-mentioned variant of the formation of chiral cyanohydrins by conversion of hydrocyanic acid with an aldehyde in the presence of an oxynitrilase as enzyme has been described, for example, by Effenberger et al. (F. Effenberger et al., Angew. Chem. 1987, 99, 491-492). The reaction shown here takes place in the 2-phase system consisting of an organic solvent phase that is not miscible with water, preferably ethyl acetate, and also an aqueous phase. The conversion is effected in this case, at least for a portion of the aldehydes, with excellent yields and optical purities. With reference to the optical purity of the cyanohydrins, the enzymatic addition of cyanide donors to aldehydes in the presence of the enzymes (R)-oxynitrilase and (S)-oxynitrilase has already been thoroughly investigated. Alternatively, the reaction may also be implemented in purely aqueous systems, with working preferably taking place at low pH values (U. Niedermeyer, M. R. Kula, Angew. Chem. 1990, 102, 423). Immobilised enzymes have also already been employed for this type of reaction (DE-PS 13 00 111). There has also been an attempt to effect the enzymatic reaction in an organic medium (P. Methe et al., U.S. Pat. No. 5,122,462; J. Am. Chem. Soc., 1999, 120, 8587; U.S. Pat. No. 5,177,242). Further conversion methods can be found in: U.S. Pat. No. 5,122,462; Biotechnol. Prog. 1999, 15, 98-104; J. Am. Chem. Soc., 1999, 120, 8587). Additionally, methods for immobilising the (S)-oxynitrilases have also been developed which in their mode of operation are comparable to those for the (R)-oxynitrilases. In this way, immobilisation of the (S)-oxynitrilases as a result of attachment to a nitrocellulose-carrier is obtained by Effenberger et al. (F. Effenberger et al., Angew. Chem. 1996, 108, 493-494). Andruski et al. give an account of immobilisation by attachment of the enzyme to a porous membrane (U.S. Pat. No. 5,177,242). Despite these, in part, thoroughly promising proposed solutions with immobilised enzymes, recently publications have again been appearing to an increasing extent that report studies with non-immobilised enzymes (for example, EP-A 0 927 766 and U.S. Pat. No. 5,714,356).

Despite the remarkable enantioselectivities that are achieved in the course of the biocatalytic asymmetric synthesis of cyanohydrin, a considerable disadvantage consists in the subsequent hydrolytic step which is needed and which is carried out “classically” via acid hydrolysis with strong mineral acids. This results in large amounts of salt refuse, constituting a problem both economically and ecologically. In addition, the hydrolysis conditions that are needed are unfavourable, since both long reaction-times of several hours and high temperatures are required. Under the hydrolysis conditions there is a high risk of racemisation.

The alternative variant of access to the desired optically active α-hydroxycarboxylic acids and amides involves—as mentioned above—an enzymatic hydrolysis of a racemic cyanohydrin.

This transformation can be catalysed by nitrilases. Nitrilases are enzymes that are able to transform organic cyano compounds into the corresponding carboxylic acids. They belong to the class E.C. 3.5.5.1 and are commercially employed, inter alia, for the synthesis of (+)-ibuprofen. An outline of the known state of the art can be found in Enzyme Catalysis in Organic Synthesis, VCH, 1995, p. 367 ff. The use of a nitrilase for preparing enantiomer-enriched mandelic acid has also been described by Yamamoto et al. (Appl. Environ. Microbiol. 1991, 57, 3028-32).

Nitrile hydratases belong to the class E.C. 4.2.1.84. They consist of α,β-subunits and may exist as multimeric polypeptides with up to 20 different units (Bunch A. W. (1998), Nitriles, in: Biotechnology, Volume 8a, Biotransformations I, chapter 6, Eds.: Rehm H. J., Reed G., Wiley-VCH, pp. 277-324; Kobayashi, M.; Shimizu, S. (1998) Metalloenzyme nitrile hydratase: structure, regulation, and application to biotechnology. Nature Biotechnology 16(8), 733-736). Many documents present the enzymatic transformation of nitrites into amides (EP 0 362 829 (Nitto); DE 44 80 132 (Institute Gniigenetika); WO 98/32872 (Novus); U.S. Pat. No. 5,200,331; DE 39 22 137; EP 0 445 646; Enzyme Catalysis in Organic Synthesis, VCH, 1995, p. 365 ff.).

However, these alternative processes also have a number of disadvantages. The enantioselectivities are often not >99% ee, which is, however, a precondition for pharmaceutical requirements in particular. In addition, there is a risk that nitrilases and nitrile hydratases could be sensitive to the presence of cyanide donors, so the starting-point has to be very pure cyanohydrins.

A general disadvantage of all previous methods is the two-stage nature of the process, resulting in a distinct reduction of the space-time yield and of the efficiency of the overall process. This two-stage process, including two reconditioning stages, was necessary, since an incompatibility of the reaction conditions of enzymatic cyanohydrin synthesis and enzymatic nitrile saponification had to be assumed.

The object of the present invention was the specification of another process for preparing enantiomer-enriched α-hydroxycarboxylic acids/amides. This process should be advantageous on a technical scale from both economic and ecological points of view. In particular, it should be superior to the processes of the state of the art with regard to costs of materials employed, robustness and efficiency (e.g. space-time yield), and should avoid the aforementioned disadvantages of the prior state of the art. In particular, the two-stage nature of the method arising previously in all processes should be avoided.

These objects are achieved in the manner specified in the claims.

By virtue of the fact that in a process for preparing enantiomer-enriched α-hydroxycarboxylic acids or enantiomer-enriched α-hydroxycarboxylic amides the starting-point is a cyanide donor, an aldehyde or ketone and the latter are caused to react in the presence of a oxynitrilase and a nitrilase or a nitrile hydratase, in extremely surprising and, according to the invention, particularly advantageous manner one arrives at the solution to the stated object. Enantiomer-enriched α-hydroxycarboxylic acids/amides can be obtained with the system according to the invention in very good yields and with particularly high enantiomer enrichments. At the time of the invention it was by no means familiar to a person skilled in the art that the enzyme cascade that has been described can be employed effectively in such a way in the existing reaction medium. In this connection it may be regarded as particularly surprising that, in particular, the considerable quantities of available cyanide did not result in the inhibition effects to be expected from the prior state of the art, particularly as regards the nitrilase or nitrile hydratase.

Accordingly, one configuration of the concrete invention relates to the fact that in a process for preparing enantiomer-enriched α-hydroxycarboxylic acids a cyanide donor is converted with an aldehyde or ketone in the presence of an oxynitrilase and a nitrilase.

Likewise, enantiomer-enriched α-hydroxycarboxylic amides can be obtained starting from a cyanide donor, an aldehyde or ketone in the presence of an oxynitrilase and a nitrile hydratase.

All the enzymes coming readily to the mind of a person skilled in the art for this purpose may be employed as oxynitrilases. A selection can be gathered from Enzyme Catalysis in Organic Synthesis, Eds.: K. Drauz, H. Waldmann, V C H, 1995, p. 580 f. The use of those which, under the given reaction conditions, bring about a long useful life and sufficient conversion is advantageous. These are, in particular, those oxynitrilases which originate from an organism selected from the group consisting of Sorghum bicolor, Hevea brasiliensis and Mannihot esculenta. For the purpose of preparing (R)-cyanohydrins, oxynitrilases from the named micro-organisms or from almond kernels are employed. In this connection it is to be noted that for the purpose of preparing (S)-α-hydroxycarboxylic acids use is preferably made of oxynitrilases of the (S)-series, and conversely, in order to be able to guarantee a sufficient conversion to the final molecule.

By way of nitrilases, in principle use may likewise be made of all those available, provided that under the given environmental conditions they guarantee a sufficient stability and conversion. A selection can be gathered from Enzyme Catalysis in Organic Synthesis, Eds.: K. Drauz, H. Waldmann, VCH, 1995, p. 365 f. These are, inter alia, those which originate from organisms that are selected from the group consisting of Rhodococcus strains or of Alcaligenes faecalis. In interaction with the reversibly acting oxynitrilase, the nitrilase brings about an irreversible conversion of the nitrile function to the carboxylic acid. By this means it is ensured that the cyanohydrin which is formed is deprived of equilibrium, leading to a complete conversion of the aldehyde or ketone or of the cyanide donor, depending on which component is employed in excess. The nitrilase should react in as highly enantioselective manner as possible, in order to ensure the desired enantiomer purity in the end product. In this case the demand on the enantioselectivity of the oxynitrilase that is employed is not so high. However, if a nitrilase is employed, the enantioselectivity of which is insufficient, importance should be attached to the presence of an appropriately differentiating oxynitrilase.

By way of nitrile hydratases, in principle use may likewise be made of all those available, provided that under the given environmental conditions they guarantee a sufficient stability and conversion. A selection can be gathered from Enzyme Catalysis in Organic Synthesis, Eds.: K. Drauz, H. Waldmann, VCH, 1995, p. 365 f. These are, inter alia, those which originate from organisms that are selected from the group consisting of Rhodococcus strains, in particular R. spec., R. rhodochrous and R. erythropolis. In this context, reference is made to EP03001715.6 and to the nitrile hydratases that are named therein and used preferentially. In interaction with the reversibly acting oxynitrilase, the nitrile hydratase brings about an irreversible conversion of the nitrile function to the carboxylic acid. By this means it is ensured that the cyanohydrin which is formed is deprived of equilibrium, leading to a complete conversion of the aldehyde or ketone or of the cyanide donor, depending on which component is employed in excess. The nitrile hydratase should react in as highly enantioselective manner as possible, in order to ensure the desired enantiomer purity in the end product. In this case the demand on the enantioselectivity of the oxynitrilase that is employed is not so high. However, if a nitrile hydratase is employed, the enantioselectivity of which is insufficient, importance should be attached to the presence of an appropriately differentiating oxynitrilase. Let it be noted that as a result of a further enzymatic or classical hydrolysis the enantiomer-enriched α-hydroxycarboxylic amides generated with this system can be converted into the corresponding acids. If in this connection an insufficient enantiomer purity should result at the stage of the amides, this can be improved by using a further amidase working enantioselectively. Suitable amidases can be found in Enzyme Catalysis in Organic Synthesis, VCH, 1995, p. 367 ff.

The aforementioned enzymes may find application in the process according to the invention both as wild type and as further developed mutants that have been improved by mutagenesis. Mutagenic processes, which are able to give rise to an improved stability and/or selectivity of the enzymes, are known to a person skilled in the art. These processes are, in particular, saturation mutagenesis, random mutagenesis, shuffling methods and also site-directed mutagenesis (Eigen M. and Gardinger W. (1984) Evolutionary molecular engineering based on RNA replication. Pure & Appl. Chem. 56(8), 967-978; Chen & Arnold (1991) Enzyme engineering for nonaqueous solvents: random mutagenesis to enhance activity of subtilisin E in polar organic media. Bio/Technology 9, 1073-1077; Horwitz, M. and L. Loeb (1986) “Promoters Selected From Random DNA Sequences” Proceedings Of The National Academy Of Sciences Of The United States Of America 83(19): 7405-7409; Dube, D. and L. Loeb (1989) “Mutants Generated By The Insertion Of Random Oligonucleotides Into The Active Site Of The Beta-Lactamase Gene” Biochemistry 28(14): 5703-5707; Stemmer PC (1994). Rapid evolution of a protein in vitro by DNA shuffling. Nature. 370; 389-391 and Stemmer PC (1994) DNA shuffling by random fragmentation and reassembly: In vitro recombination for molecular evolution. Proc Natl Acad Sci USA. 91; 10747-10751). The term ‘improved selectivity’ is to be understood to mean, according to the invention, an increase in the enantioselectivity and/or a reduction in the substrate selectivity.

The enzyme being considered in the given case can be used for the application in free form, as a homogeneously purified compound. Furthermore, the enzyme may also be employed as a constituent of an intact guest organism or in conjunction with the decomposed and arbitrarily highly purified cell mass of the host organism. Also possible is the use of the enzymes in immobilised form (Bhavender P. Sharma, Lorraine F. Bailey and Ralph A. Messing, “Immobilisierte Biomaterialien—Techniken und Anwendungen”, Angew. Chem. 1982, 94, 836-852). Immobilisation is advantageously effected by lyophilisation (Dordick et al. J. Am. Chem. Soc. 194, 116, 5009-5010; Okahata et al. Tetrahedron Lett. 1997, 38, 1971-1974; Adlercreutz et al. Biocatalysis 1992, 6, 291-305). Lyophilisation in the presence of surface-active substances such as Aerosol OT or polyvinyl pyrrolidone or polyethylene glycol (PEG) or Brij 52 (diethylene glycol monocetyl ether) (Goto et al. Biotechnol. Techniques 1997, 11, 375-378) is quite particularly preferred. Use as CLECs is also conceivable (St Clair et al. Angew Chem Int Ed Engl 2000 January, 39(2), 380-383).

In principle, the concrete process of the invention may be implemented in purely aqueous solution. However, it is also possible to add arbitrary portions of a water-soluble organic solvent to the aqueous solution, in order, for example, to optimise the reaction with regard to sparingly water-soluble substrates. Ethylene glycol, DME or glycerin come into consideration in particular as such solvents. But multi-phase systems, in particular two-phase systems, exhibiting an aqueous phase as solvent mixture may, furthermore, also serve for the process according to the invention. Here the use of certain solvents that are not soluble in water has already proved worthwhile (DE 10233107). The statements made therein in this regard apply here correspondingly.

In principle, a person skilled in the art is free in the choice of the temperature prevailing during the reaction. Such a person is preferably guided by the receipt of as high a yield of product as possible in the highest possible purity and in the shortest possible time. In addition, the enzymes that are employed should be sufficiently stable at the temperatures that are employed, and the reaction should proceed with as high an enantioselectivity as possible. With regard to the use of enzymes derived from thermophilic organisms, temperatures of 80-100° C. may definitely represent the upper limit of the temperature range in the course of the reaction. As a lower limit in aqueous systems, temperatures of −15° C. are certainly sensible. Advantageously, a temperature interval should be adjusted between 10° C. and 60° C., particularly preferably between 20° C. and 40° C.

The pH value during the reaction is ascertained by a person skilled in the art on the basis of the enzyme stabilities and rates of conversion, and is appropriately adjusted for the process according to the invention. In general, the preferred range for enzymes will be chosen from pH 3 to 11. A pH range from 3.0 to 10.0, in particular 6.0 to 9.0, may preferably obtain.

In a further configuration the invention relates to an enzymatic reaction system exhibiting an oxynitrilase, a nitrilase or nitrile hydratase, water, a cyanide donor and an aldehyde or a ketone. Optionally in addition, the presence of an organic solvent may be possible, as has been described in detail above.

In principle, the same advantages and preferred embodiments apply in respect of this reaction system as have already been stated with reference to the process according to the invention.

The reaction system is advantageously employed, for example, in a stirred tank, in a stirred-tank cascade or in membrane reactors that can be operated both in batch operation and continuously.

Within the scope of the invention the term ‘membrane reactor’ is to be understood to mean any reaction vessel in which the catalyst is enclosed in a reactor while low-molecular substances are supplied to the reactor or are able to leave it. In this connection the membrane may be integrated directly into the reaction chamber or may be incorporated outside in a separate filtration module, with the reaction solution flowing continuously or intermittently through the filtration module, and with the retentate being recirculated into the reactor. Suitable embodiments are described, inter alia, in WO 98/22415 and in Wandrey et al. in Jahrbuch 1998, Verfahrenstechnik und Chemieingenieurwesen, VDI p. 151 ff.; Wandrey et al. in Applied Homogeneous Catalysis with Organometallic Compounds, Vol. 2, VCH 1996, p. 832 ff.; Kragl et al., Angew. Chem. 1996, 6, 684 f.

The continuous mode of operation which is possible in this apparatus in addition to the batch and semicontinuous modes of operation may, as desired, be implemented in the cross-flow filtration mode (FIG. 1) or as dead-end filtration (FIG. 2). Both process variants are described in principle in the state of the art (Engineering Processes for Bioseparations, Ed.: L. R. Weatherley, Heinemann, 1994, 135-165; Wandrey et al., Tetrahedron Asymmetry 1999, 10, 923-928).

A further aspect of the invention is constituted by a whole-cell catalyst exhibiting a cloned gene for an oxynitrilase and a nitrilase or a nitrile hydratase. The whole-cell catalyst according to the invention should preferably exhibit one of the aforementioned representatives by way of oxynitrilase or alternatively nitrilase or nitrile hydratase. In the case where a nitrile hydratase is present, the whole-cell catalyst preferably likewise contains a cloned gene for an amidase. The preparation of such an organism is familiar to a person skilled in the art (PCT/EP00/08473; PCT/US00/08159; Sambrook et al. 1989, Molecular cloning: A Laboratory Manual, 2nd Edition, Cold Spring Harbor Laboratory Press, Balbas P & Bolivar F. 1990; Design and construction of expression plasmid vectors in E. coli, Methods Enzymology 185, 14-37; Vectors: A Survey of Molecular Cloning Vectors and Their Uses. R. L. Rodriguez & D. T. Denhardt, Eds: 205-225). The processing modes stated therein may be put into effect here in equivalent manner. With respect to the general procedure (PCR, cloning, expression etc.), reference may also be made to the following literature and respective citations therein: Universal GenomeWalker™ Kit User Manual, Clontech, 3/2000 and literature cited therein; Triglia T.; Peterson, M. G. and Kemp, D. J. (1988), A procedure for in vitro amplification of DNA segments that lie outside the boundaries of known sequences, Nucleic Acids Res. 16, 8186; Sambrook, J.; Fritsch, E. F. and Maniatis, T. (1989), Molecular cloning: a laboratory manual, 2^{nd} ed., Cold Spring Harbor Laboratory Press, New York; Rodriguez, R. L. and Denhardt, D. T. (eds) (1988), Vectors: a survey of molecular cloning vectors and their uses, Butterworth, Stoneham.

The advantage of such an organism is the simultaneous expression of both enzyme systems, by virtue of which only one rec organism has to be reared for the reaction. In order to match the expression of the enzymes with regard to their rates of conversion, the appropriately coding nucleic-acid fragments may be accommodated on different plasmids with different copy-numbers, and/or use may be made of variably strong promoters for a variably strong expression of the genes. With enzyme systems that have been matched in such a way, advantageously an accumulation of an intermediate compound, acting in appropriate circumstances in inhibiting manner, does not arise, and the reaction under consideration can proceed at an optimal overall rate. This is, however, sufficiently known to a person skilled in the art (PCT/EP00/08473; Gellissen et al,. Appl. Microbiol. Biotechnol. 1996, 46, 46-54). By way of micro-organisms, in principle use may be made of all organisms coming into consideration for this purpose by a person skilled in the art, such as, for example, yeasts such as Hansenula polymorpha, Pichia sp., Saccharomyces cerevisiae, prokaryotes, such as E. coli, Bacillus subtilis or eukaryotes such as mammalian cells, insect cells. Strains of E. coli should preferably be used for this purpose. Quite particularly preferred are: E. coli XL1 Blue, NM 522, JM101, JM109, JM105, RR1, DH5α, TOP10⁻ or HB101. In extremely preferred manner, by way of organism use may be made of that named in DE 101 55 928.

By way of aldehydes or ketones, use may be made of those having aliphatic or aromatic/heteroaromatic residues. These may be arbitrarily branched and/or substituted, provided that these residues prove to be inert as regards the actual conversion. Advantageously, compounds of the general formula (I) are employed in the reaction. 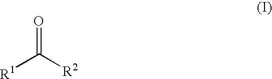
in which

R¹ may signify (C₁-C₈)-alkyl, (C₂-C₈)-alkenyl, (C₂-C₈)-alkinyl, (C₁-C₈)-alkoxyalkyl (C₃-C₈)-cycloalkyl, (C₆-C₁₈)-aryl, (C₇-C₁₉)-aralkyl, (C₃-C₁₈)-heteroaryl, (C₄-C₁₉)-heteroaralkyl, ((C₁-C₈)-alkyl)₁₋₃-(C₃-C₈)-cycloalkyl, ((C₁-C₈)-alkyl)₁₋₃-(C₆-C₁₈)-aryl, ((C₁-C₈)-alkyl)₁₋₃-(C₃-C₁₈) )-heteroaryl and

R² may signify H, R¹.

By way of cyanide donors, all the compounds available to a person skilled in the art under the given circumstances come into consideration. In particular, those are employed which can be obtained as inexpensively as possible, whereby, however, importance is to be attached to an optimal conversion of these compounds in the reaction according to the invention. Cyanide donors are, by definition, compounds that permit CN⁻ ions to be released under the given reaction conditions. In particular, these are those selected from the group containing hydrocyanic acid, metal cyanides such as alkali cyanides, trimethylsilyl cyanide.

In general, in the reaction according to the invention the procedure is such that the enzymes as such (wild type, prepared by recombinant means), as biomass or in the intact guest organism (e.g. whole-cell catalyst), are charged together with the aldehyde or ketone in an aqueous reaction matrix, and subsequently the cyanide donor, such as, for example, an alkali cyanide (sodium cyanide), is added. Under the appropriate reaction conditions the corresponding cyanohydrin is formed straightaway by way of intermediate, and the enantiomer-enriched α-hydroxycarboxylic acid or amide is formed therefrom. These may be isolated from the reaction mixture in accordance with the process known to a person skilled in the art. This is preferably done in such a way that the relatively high-molecular-weight constituents are removed by filtration and the acid or amide is either isolated from the mixture immediately by crystallisation or, in the case of a lipophilic acid or amide, a step of extraction into an organic medium is interpolated prior to isolation. A reconditioning of the acid by means of ion-exchange chromatography is also possible.

In such a way, benzaldehyde, for example, can be transformed with sodium cyanide into the corresponding mandelic acid in high yields of >80%, preferably >85%, still more preferably >90%, 91%, 92%, 93%, 94%, further preferred >95%, 96%, 97% and with enantiomer enrichments of >90%, 91%, 92%, 93%, 94%, further preferred >95%, 96%, 97% and, extremely preferred, >98%, 99%.

With a view to preparing the whole-cell catalyst according to the invention, a person skilled in the art will make use of the previously described methods of the state of the art. In detail, a nitrilase or nitrile hydratase and also an oxynitrilase are contained in such a whole-cell catalyst. The sequences of the relevant genes can be gathered from publicly accessible gene databanks, for example from the NCBI gene databank (Internet: http://www.ncbi.nlm.nih.gov/Genbank/GenbankOverview.html). Particularly preferred in this connection are enzymes, particularly nitrilases or nitrile hydratases, having a high cyanide resistance. In this connection the procedure is preferably such that the corresponding sequences are ligated jointly with the corresponding necessary gene sequences such as promoters etc. either into a plasmid or onto several plasmids. After this, said plasmids are transformed into the selected organism, the latter is replicated, and active clone is then inserted—intact or in the form of crushed biomass—into the reaction. At the time of the invention it was by no means obvious that in such a manner a conversion as described, with such good results, is possible.

To be regarded as (C₁-C₈)-alkyl are methyl, ethyl, n-propyl, isopropyl, n-butyl, isobutyl, sec-butyl, tert-butyl, pentyl, hexyl, heptyl or octyl together with all the bond isomers. These may be monosubstituted or polysubstituted with (C₁-C₈)-alkoxy, (C₁-C₈)-haloalkyl, OH, halogen, NH₂, NO₂, SH, S—(C₁-C₈)-alkyl.

The term ‘(C₂-C₈)-alkenyl’ is to be understood to mean, with the exception of methyl, a (C₁-C₈)-alkyl residue as presented above which exhibits at least one double bond.

The term ‘(C₂-C₈)-alkinyl’ is to be understood to mean, with the exception of methyl, a (C₁-C₈)-alkyl residue as presented above which exhibits at least one triple bond.

The term ‘(C₃-C₈)-cycloalkyl’ is to be understood to mean cyclopropyl, cyclobutyl, cyclopentyl, cyclohexyl or cycloheptyl residues etc. These may be substituted with one or more halogens and/or residues containing N, O, P, S atoms and/or may exhibit residues containing N, O, P, S atoms in the ring, such as, for example, 1-, 2-, 3-, 4-piperidyl, 1-, 2-, 3-pyrrolidinyl, 2-, 3-tetrahydrofuryl, 2-, 3-, 4-morpholinyl. The latter may be monosubstituted or polysubstituted with (C₁-C₈)-alkoxy, (C₁-C₈)-haloalkyl, OH, halogen, NH₂, NO₂, SH, S—(C₁-C₈)-alkyl, (C₁-C₈)-alkyl.

The term ‘(C₆-C₁₈)-aryl residue’ is to be understood to mean an aromatic residue with 6 to 18 C atoms. These include, in particular, compounds such as phenyl, naphthyl, anthryl, phenanthryl, biphenyl residues. The latter may be monosubstituted or polysubstituted with (C₁-C₈)-alkoxy, (C₁-C₈)-haloalkyl, OH, halogen, NH₂, NO₂, SH, S—(C₁-C₈)-alkyl, (C₁-C₈)-alkyl.

A (C₇-C₁₉)-aralkyl residue is a (C₆-C₁₈)-aryl residue that is bonded to the molecule via a (C₁-C₈)-alkyl residue.

(C₁-C₈)-alkoxy is a (C₁-C₈)-alkyl residue that is bonded to the molecule under consideration via an oxygen atom.

(C₁-C₈)-haloalkyl is a (C₁-C₈)-alkyl residue substituted with one or more halogen atoms.

A (C₃-C₁₈)-heteroaryl residue denotes, within the scope of the invention, a five-, six- or seven-membered aromatic ring system consisting of 3 to 18 C atoms which exhibits heteroatoms such as, for example, nitrogen, oxygen or sulfur in the ring. Regarded as such heteroaromatics are, in particular, residues such as 1-, 2-, 3-furyl, such as 1-, 2-, 3-pyrrolyl, 1-, 2-, 3-thienyl, 2-, 3-, 4-pyridyl, 2-, 3-, 4-, 5-, 6-, 7-indolyl, 3-, 4-, 5-pyrazolyl, 2-, 4-, 5-imidazolyl, acridinyl, quinolinyl, phenanthridinyl, 2-, 4-, 5-, 6-pyrimidinyl. The latter may be monosubstituted or polysubstituted with (C₁-C₈)-alkoxy, (C₁-C₈)-haloalkyl, OH, halogen, NH₂, NO₂, SH, S—(C₁-C₈)-alkyl, (C₁-C₈)-alkyl.

The term ‘(C₄-C₁₉)-heteroaralkyl’ is to be understood to mean a heteroaromatic system corresponding to the (C₇-C₁₉)-aralkyl residue.

Fluorine, chlorine, bromine and iodine come into consideration as halogens.

The term ‘enantiomer-enriched’ denotes the fact that one optical antipode is present in a mixture with its other one in a proportion amounting to >50%.

The structures that have been presented relate, in the case where one stereocentre is present, to both possible enantiomers and, in the case where more than one stereocentre is present in the molecule, to all possible diastereomers and, with respect to a diastereomer, to the possible two enantiomers of the compound in question which are included thereunder.

The stated passages from the literature are to be regarded as being encompassed by the disclosure of this invention.

1-11. (canceled)

12. A method for preparing an enantiomer-enriched α-hydroxycarboxylic acid or an enantiomer-enriched α-hydroxycarboxylic amide, comprising reacting a cyanide donor with an aldehyde or ketone in the presence of an oxynitrilase and either a nitrilase or a nitrite hydratase.

13. The method of claim 12, wherein, said cyanide donor is hydrogen cyanide.

14. The method of claim 12, wherein said cyanide donor is reacted with said aldehyde or ketone in the presence of an oxynitrilase and a nitrilase.

15. The method of claim 12, wherein said cyanide donor is reacted with said aldehyde or ketone in the presence of an oxynitrilase and nitrile hydratase.

16. The method of claim 15, wherein, in addition to said oxynitrilase and nitrile hydratase, an amidase is present.

17. The process of claim 12, wherein said oxynitrilase is selected from the group consisting of Sorghum bicolor, Hevea brasiliensis, Mannihot esculenta and almond kernels.

18. The process claim 14, wherein said nitrilase is from an organism selected from either a strain of Rhodococcus or Alcaligenes faecalis.

19. The process of claim 15, wherein said nitrile hydratase is from an organism selected from the group consisting of: Rhodococcus spec., Rhodococcus rhodochrous and Rhodococcus erythropolis.

20. The process of claim 12, wherein said cyanide donor is reacted with said aldehyde or ketone at a temperature of 20-40° C.

21. The process of claim 12, wherein:
a) said oxynitrilase is selected from the group consisting of Sorghum bicolor, Hevea brasiliensis, Mannihot esculenta and almond kernels;
b) said nitrilase is from an organism selected from either a strain of Rhodococcus or Alcaligenes faecalis; and
c) said nitrile hydratase is from an organism selected from the group consisting of: Rhodococcus spec., Rhodococcus rhodochrous and Rhodococcus erythropolis.

22. An enzymatic reaction composition comprising an oxynitrilase, a nitrilase or a nitrile hydratase, water, a cyanide donor and an aldehyde or a ketone.

23. The enzymatic reaction composition of claim 22, wherein said composition comprises an oxynitrilase, a nitrilase, water, a cyanide donor and an aldehyde or a ketone.

24. The enzymatic reaction composition of claim 22, wherein wherein said composition comprises an oxynitrilase, a nitrile hydratase, water, a cyanide donor and an aldehyde or a ketone.

25. The enzymatic reaction composition of claim 24, further comprising an amidase.

26. The enzymatic reaction composition of claim 22, wherein:
a) said oxynitrilase is selected from the group consisting of Sorghum bicolor, Hevea brasiliensis, Mannihot esculenta and almond kernels;
b) said nitrilase is from an organism selected from either a strain of Rhodococcus or Alcaligenes faecalis; and
c) said nitrile hydratase is from an organism selected from the group consisting of: Rhodococcus spec., Rhodococcus rhodochrous and Rhodococcus erythropolis.

27. A cell capable of serving as a whole-cell catalyst, wherein said cell comprises a cloned gene encoding an oxynitrilase and a cloned gene encoding either a nitrilase or a nitrile hydratase.

28. The cell of claim 27, wherein said cell comprises a cloned gene encoding an oxynitrilase and a cloned gene encoding a nitrilase.

29. The cell of claim 27, wherein said cell comprises a cloned gene encoding an oxynitrilase and a cloned gene encoding nitrile hydratase.

30. The cell of claim 29, further comprising a cloned gene encoding an amidase.

31. The cell of claim 27, wherein:
a) said oxynitrilase is selected from the group consisting of Sorghum bicolor, Hevea brasiliensis, Mannihot esculenta and almond kernels;
b) said nitrilase is from an organism selected from either a strain of Rhodococcus or Alcaligenes faecalis; and
c) said nitrile hydratase is from an organism selected from the group consisting of: Rhodococcus spec., Rhodococcus rhodochrous and Rhodococcus erythropolis.
